(19) United States
(12) Patent Application Publication
(10) Pub. No.: US 2024/0164920 A1
(43) Pub. Date: May 23, 2024

(54) STENT WITH SELECTIVE MEMBRANE COATING

(71) Applicant: BOSTON SCIENTIFIC SCIMED, INC., Maple Grove, MN (US)

(72) Inventors: Martyn G. Folan, Galway (IE); Peter L. Dayton, Brookline, MA (US)

(73) Assignee: BOSTON SCIENTIFIC SCIMED, INC., Maple Grove, MN (US)

(21) Appl. No.: 18/517,961

(22) Filed: Nov. 22, 2023

Related U.S. Application Data
(60) Provisional application No. 63/427,618, filed on Nov. 23, 2022.

(51) Int. Cl. A61F 2/90

(52) U.S. Cl. CPC A61F 2/90; A61F 2002/0086

(57) ABSTRACT

Medical devices and methods for using medical devices are disclosed. An example medical device includes a scaffold including an inner surface, an outer surface, a proximal end region, a distal end region, a lumen extending from the proximal end region to the distal end region, and a retention member extending radially away from the outer surface. The retention member has a distally facing surface and a proximally facing surface. A membrane is disposed within the lumen of the tubular scaffold and is secured to the inner surface of the tubular scaffold at a first circumferential attachment region and at a second circumferential attachment region. Further, the membrane is unattached to the inner surface of the tubular scaffold between the first circumferential attachment region and the second circumferential attachment region to define a tissue ingrowth region between the inner surface of the tubular scaffold and an outwardly-facing surface of the membrane.

### CROSS-REFERENCE TO RELATED APPLICATIONS

This application claims the benefit of U.S. Provisional Patent Application Ser. No. 63/427,618 filed on Nov. 23, 2022, the disclosure of which is incorporated herein by reference.

### TECHNICAL FIELD

The present disclosure pertains to medical devices, methods for manufacturing medical devices, and uses thereof. More particularly, the present disclosure pertains to stents having selective membrane coatings for implantation in body lumens, and associated methods.

### BACKGROUND

Implantable medical devices (e.g., expandable stents) may be designed to treat a variety of medical conditions in the body. For example, some expandable stents may be designed to radially expand and support a body lumen and/or provide a fluid pathway for digested material, blood, or other fluid to flow therethrough following a medical procedure. Some medical devices may include radially or self-expanding stents which may be implanted transluminally via a variety of medical device delivery systems. These stents may be implanted in a variety of body lumens such as coronary or peripheral arteries, the esophageal tract, gastrointestinal tract (including the intestine, stomach and the colon), tracheobronchial tract, urinary tract, biliary tract, vascular system, etc.

In some instances it may be desirable to design stents to include sufficient flexibility while maintaining sufficient radial force to open the body lumen at the treatment site. However, in some stents, the compressible and flexible properties that assist in stent delivery may also cause a stent to migrate from its originally deployed position. For example, stents that are designed to be positioned in the gastrointestinal and/or biliary tract may migrate due to peristalsis (i.e., the involuntary constriction and relaxation of the muscles of the stomach, intestine, and colon). Further, the generally moist and inherently lubricious environment of the stomach, intestine, colon, etc. may contribute to a stent's tendency to migrate when deployed therein. Further yet, the relative motion of non-connected structures (e.g., the relative motion of a hepatic duct and the stomach) may contribute to a stent's tendency to migrate when deployed therein.

Various medical procedures involve the temporary or permanent joining of non-connected anatomical structures. Some examples include a hepaticogastrostomy (HGS), which involves joining a hepatic duct and the stomach to drain the bile duct, EUS-guided gallbladder drainage (EUS-GBD), utilized for the treatment of acute cholecystitis and symptomatic cholelithiasis in patients who are poor operative candidates, a gastrojejunal(GJ) bypass or gastrojejunostomy procedure to create an anastomosis between the small intestine and stomach wall, and stomas to create an artificial opening into the large intestine or other region of the digestive tract. In these medical procedures, peristalsis and gross organ movement in one or both of the anatomical structures being connected may create difficulties in using stents to join the structures due to stent migration. Accordingly procedures may require a stent to permit leak-free drainage from one anatomical structure (e.g., hepatic ducts) to another anatomical structure (e.g., stomach) while also permitting tissue ingrowth into the stent to prevent stent migration.

Therefore, it may be desirable to design a stent having both drainage capabilities and anti-migration features to reduce the stent's tendency to migrate. Examples of medical devices including both drainage and anti-migration features, and methods of using them are disclosed herein.

### SUMMARY

This disclosure provides design, material, manufacturing method, and use alternatives for medical devices. An example expandable medical device includes a tubular scaffold, the tubular scaffold including an inner surface, an outer surface, a proximal end region, a distal end region, a lumen extending from the proximal end region to the distal end region, and a retention member extending radially away from the outer surface, wherein the retention member has a distally facing surface and a proximally facing surface. The medical device also includes a membrane disposed within the lumen of the tubular scaffold. The membrane is secured to the inner surface of the tubular scaffold at a first circumferential attachment region, and the membrane is secured to the inner surface of the tubular scaffold at a second circumferential attachment region. Further, the membrane is unattached to the inner surface of the tubular scaffold between the first circumferential attachment region and the second circumferential attachment region to define a tissue ingrowth region between the inner surface of the tubular scaffold and an outwardly-facing surface of the membrane.

Alternatively or additionally to the embodiment above, wherein the membrane is configured to maintain a passageway therethrough.

Alternatively or additionally to the embodiment above, wherein the tissue ingrowth region extends circumferentially around the inner surface of the tubular scaffold.

Alternatively or additionally to the embodiment above, wherein the membrane is formed from an elastic material.

Alternatively or additionally to the embodiment above, wherein the membrane is designed to permit tissue ingrowth between the inner surface of the tubular scaffold and an outwardly-facing surface of the membrane.

Alternatively or additionally to the embodiment above, wherein the first circumferential attachment region is secured to the inner surface of the tubular scaffold along the distal end region and wherein the second circumferential attachment region is secured to the inner surface of the tubular scaffold at a position distal to the retention member.

Alternatively or additionally to the embodiment above, wherein the tubular scaffold includes interstices extending from the outer surface of the tubular scaffold to the inner surface of the tubular scaffold, and wherein the membrane spans the interstices of the portion of the tubular scaffold which defines the retention member.

Alternatively or additionally to the embodiment above, wherein the tubular scaffold includes interstices extending from the outer surface of the tubular scaffold to the inner surface of the tubular scaffold, and wherein the membrane encapsulates the interstices of the portion of the tubular scaffold which defines the retention member.

Alternatively or additionally to the embodiment above, wherein the first circumferential attachment region is secured to the inner surface of the tubular scaffold along the distal end region and wherein the second circumferential attachment region is secured to the inner surface of the tubular scaffold at a position proximal to the retention member.

Alternatively or additionally to the embodiment above, wherein the tubular scaffold includes interstices extending from the outer surface of the tubular scaffold to the inner surface of the tubular scaffold, wherein tissue is permitted to grow through the interstices of the portion of the tubular scaffold between the first circumferential attachment region and the second circumferential attachment region.

Alternatively or additionally to the embodiment above, wherein the distal end region of the tubular scaffold further includes a flared portion.

Alternatively or additionally to the embodiment above, wherein the first circumferential attachment region is secured to the inner surface of the tubular scaffold at a position proximal to the flared portion and wherein the second circumferential attachment region is secured to the inner surface of the tubular scaffold at a position distal to the retention member.

Alternatively or additionally to the embodiment above, wherein the membrane is in direct contact with the inner surface of the portion of the tubular scaffold defining the retention member.

Alternatively or additionally to the embodiment above, wherein the flared portion includes interstices extending from the outer surface of the tubular scaffold to the inner surface of the tubular scaffold, wherein the flared portion is devoid of the membrane such that tissue is permitted to grow through the interstices of the tubular scaffold along the flared portion.

Alternatively or additionally to the embodiment above, wherein the retention member has a diameter, wherein the flared portion has a diameter, and wherein the diameter of the retention member is greater than the diameter of the flared portion.

Alternatively or additionally to the embodiment above, wherein the distally facing surface of the retention member is substantially parallel to the proximally facing surface of the retention member.

Alternatively or additionally to the embodiment above, wherein the membrane is further secured to the inner surface of the tubular scaffold at a plurality of spaced-apart discrete attachment points positioned between the first circumferential attachment region and the second circumferential attachment region.

Alternatively or additionally to the embodiment above, wherein regions of the membrane between the plurality of spaced-apart discrete attachment points are radially spaced from the inner surface of the tubular scaffold, creating a plurality of tissue ingrowth regions positioned between the first circumferential attachment region and the second circumferential attachment region.

Another expandable medical device includes a tubular scaffold, the tubular scaffold including an inner surface, an outer surface, a proximal end region, a distal end region including a flared portion, a lumen extending from the proximal end region to the distal end region, and a retention member extending radially away from the outer surface, wherein the retention member has a distally facing surface positioned substantially parallel to a proximally facing surface. The medical device also includes a membrane disposed within the lumen of the tubular scaffold, and wherein the membrane is secured to the inner surface of the tubular scaffold at a first circumferential attachment region, and wherein the membrane is secured to the inner surface of the tubular scaffold at a second circumferential attachment region and wherein the membrane is configured to maintain a passageway therethrough.

Another expandable medical device includes a tubular scaffold, the tubular scaffold including an inner surface, an outer surface, a proximal end region, a distal end region including a flared portion, a lumen extending from the proximal end region to the distal end region, and a retention member extending radially away from the outer surface, wherein the retention member has a distally facing surface positioned substantially parallel to a proximally facing surface. The medical device also includes a membrane disposed within the lumen of the tubular scaffold, and wherein the membrane is secured to the inner surface of the tubular scaffold at a first circumferential attachment region, and wherein the membrane is secured to the inner surface of the tubular scaffold at a second circumferential attachment region. Further the flared portion includes interstices extending from the outer surface of the tubular scaffold to the inner surface of the tubular scaffold, wherein the flared portion is devoid of the membrane such that tissue is permitted to grow through the interstices of the tubular scaffold along the flared portion.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description of various embodiments in connection with the accompanying drawings, in which:

FIG. 1 illustrates a region of the digestive tract;

FIG. 2 illustrates an example stent including a flared portion;

FIG. 3 is a cross-sectional view of the stent of FIG. 2 including an inner membrane;

FIG. 4 is an alternative cross-sectional view of the stent of FIG. 2 including an inner membrane;

FIG. 5 is an alternative cross-sectional view of the stent of FIG. 2 including an inner membrane;

FIG. 6 is an alternative cross-sectional view of the stent of FIG. 2 including an inner membrane;

FIG. 7 is an alternative cross-sectional view of the stent of FIG. 2 including an inner membrane;

FIG. 8 is an alternative cross-sectional view of the stent of FIG. 2 including an inner membrane;

FIG. 9 is a cross-sectional view of the example stent taken along line 9-9 of FIG. 8;

FIG. 10 illustrates an example stent positioned in a portion of the digestive tract;

FIG. 11 is an illustration of another example stent in a relaxed configuration;

FIG. 12 is an illustration of the example stent of FIG. 11 in an elongated configuration;

FIG. 13 is a cross-sectional view of the stent shown in FIG. 11;

FIG. 14 is a cross-sectional view of the stent shown in FIG. 12;

FIG. 15 is an illustration of another example stent in a relaxed configuration; and

FIG. 16 is a cross-sectional view of the example stent of FIG. 15 in an elongated configuration.

While the disclosure is amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the scope of the disclosure.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (i.e., having the same function or result). In many instances, the term “about” may be indicative as including numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

Although some suitable dimensions ranges and/or values pertaining to various components, features and/or specifications are disclosed, one of skill in the art, incited by the present disclosure, would understand desired dimensions, ranges and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following detailed description should be read with reference to the drawings in which similar elements in different drawings are numbered the same. The detailed description and the drawings, which are not necessarily to scale, depict illustrative embodiments and are not intended to limit the scope of the disclosure. The illustrative embodiments depicted are intended only as exemplary. Selected features of any illustrative embodiment may be incorporated into an additional embodiment unless clearly stated to the contrary.

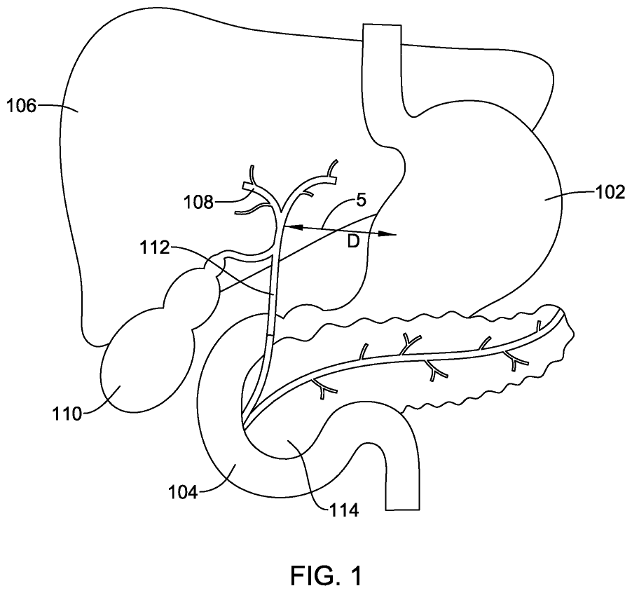

FIG. 1 illustrates various organs in the digestive tract, including the stomach 102, duodenum 104, liver 106, hepatic duct 108, gallbladder 110, common bile duct 112, and pancreas 114.

Bile, which is produced in the liver 106, flows through a series of hepatic ducts 108 that drain into one large duct called the common bile duct (CBD) 112. The CBD then connects to the duodenum 104, allowing the bile to flow into the duodenum for digestion. If the hepatic or bile ducts become blocked, bile cannot drain normally and backs up or builds up in the liver 106. Blocked bile ducts may cause jaundice, dark urine, nausea and poor appetite, leading to potentially serious conditions.

Endoscopic retrograde cholangiopancreatography (ERCP) may be used to diagnose and treat conditions of the bile ducts, including, for example, gallstones, inflammatory strictures, leaks (e.g., from trauma, surgery, etc.), and cancer. Blockage of the biliary duct may occur in many of the disorders of the biliary system, including the disorders of the liver, such as, primary schlerosing cholangitis, stone formation, scarring in the duct, etc. Draining blocked fluids from the biliary system may be used to treat the disorders. Methods of biliary drainage include the placement of plastic or metal stents to relieve the blockage. In the case of a gallstone causing the obstruction in the duct, a number of products are also available to resolve this through ERCP. However, access to the bile ducts via ERCP may not be possible due to a variety of reasons such as a tumor blocking the passageway, anatomic variation, periampullary diverticula, etc.

When ERCP methods prove unsuccessful, percutaneous drainage (PTCD) can be performed. However, PTCD may be associated with complications such as bleeding and bile leakage. If subsequent internal drainage cannot be achieved, the patient would have to accept long-term external biliary drainage, which can be uncomfortable and have significant impairment of quality of life.

Endoscopic Ultrasound (EUS) guided biliary drainage (BD) offers an alternative option to surgery and percutaneous drainage for treating obstructive jaundice when ERCP drainage fails. Hepaticogastrostomy (HGS) may be performed to join the hepatic duct 108 to the stomach 102. This would allow the build-up of bile to flow into the stomach and may relieve the symptoms caused by bile buildup, i.e. jaundice. However, the hepatic duct 108 and the stomach 102 are spaced apart by a distance D indicated by the arrow 5 in FIG. 1. The distance D between the organs may require a relatively long stent. Additionally, as the stomach muscles contract to churn food, the distance D between the gastric wall of the stomach 102 relative to the hepatic duct 108 varies, from a relatively small distance D when the stomach is relaxed as to a greater distance when the stomach is contracted. In addition to the gastric wall flexing, the stomach undergoes peristalsis during digestion. This relative motility of the stomach is understood to be complex, in three dimensions rather than in an exclusively linear manner. The distance between target organs to be joined, the relative movement of at least one of the organs, as well as the normal motion of the body (e.g., twisting, running, jumping, etc.) may increase the chance of stent migration.

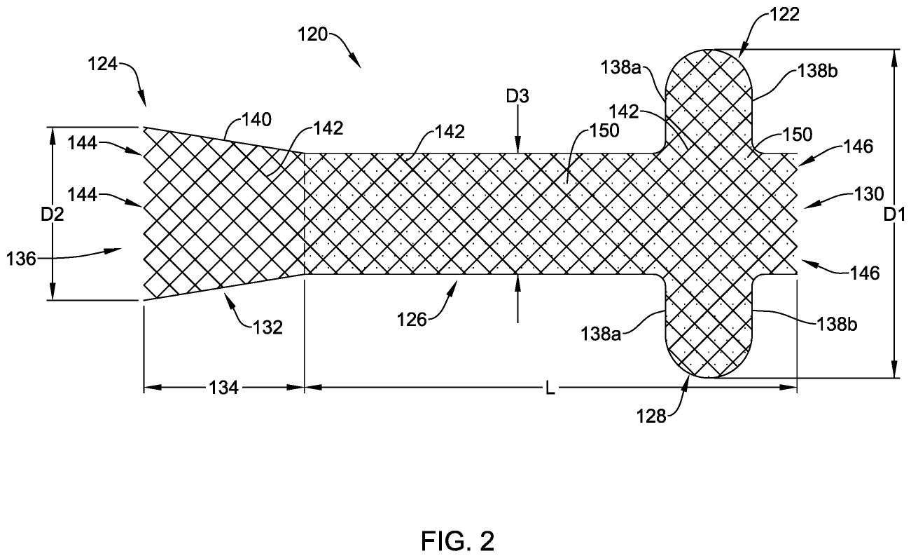

FIG. 2 illustrates an example expandable medical device, namely a stent 120 (e.g., drainage stent) including a first end region 122 (e.g., a proximal end region), a second end region 124 (e.g., distal end region) and a medial region 126 extending between the first end region 122 and the second end region 124. The stent 120 may include one or more stent strut members 142 forming a tubular scaffold. Stent strut members 142 may extend helically, longitudinally, circumferentially, or otherwise along stent 120. While FIG. 2 shows stent strut members 142 extending along the entire length of stent 120, in other examples, the stent strut members 142 may extend only along a portion of stent 120.

In some examples, the stent 120 may be a self-expanding stent. Self-expanding stent examples may include stents having one or more strut members 142 combined to form a rigid and/or semi-rigid tubular stent scaffold. For example, the stent strut members 142 of the stent 120 may include wires or filaments which are braided, wrapped, intertwined, interwoven, weaved, knitted, looped (e.g., bobbinet-style) or the like to form the tubular scaffold. For example, while the example stents disclosed herein may resemble a braided stent, this is not intended to limit the possible stent configurations. Rather, the stents depicted in the Figures may be stents that are braided, knitted, wrapped, intertwined, interwoven, weaved, looped (e.g., bobbinet-style) or the like to form the stent scaffold. In various embodiments, the woven, braided and/or knitted member(s) may include a single filament woven upon itself, or multiple filaments woven together. In various embodiments, any of the woven, braided and/or knitted member(s), which comprise the elongate tubular body, may include a variety of different cross-sectional shapes (e.g., oval, round, flat, square, etc.).

Alternatively, the stent 120 may be a monolithic structure formed from a cylindrical tubular member, such as a single, cylindrical tubular laser-cut Nitinol tubular member, in which the remaining portions of the tubular member form the strut members 142. Openings or interstices through the wall of the stent 120 may be defined between adjacent strut members 142.

The stent 120 in the examples disclosed herein may be constructed from a variety of materials. For example, the stent 120 (e.g., self-expanding or balloon expandable) may be constructed from a metal (e.g., Nitinol, Elgiloy, etc.). In other instances, the stent 120 may be constructed from a polymeric material (e.g., PET). In yet other instances, the stent 120 may be constructed from a combination of metallic and polymeric materials. Additionally, stent 120 may include a bioabsorbable and/or biodegradable material.

Additionally, the stent 120 may be configured to shift between a first (e.g., constrained, collapsed, non-expanded) configuration and a second (e.g., non-constrained, expanded) configuration. In an expanded configuration, the first end region 122 of the stent 120 may include a retention member 128 defining a first opening 130. The retention member 128 may be formed from the stent strut members 142 used to form other portions of the stent 120. For example, the retention member 128 may be formed from the same stent strut members 142 used to form the medial region 126.

FIG. 2 further illustrates that, in an expanded configuration, the second end region 124 of the stent 120 may include a flared portion 132 having a second opening 136. In some examples, the stent 120 may not include a flared portion 132. Rather, in some examples, the medial region 126 may include a uniform outer diameter along its length.

Additionally, the medial region 126 of the stent 120 may include a circumference and a longitudinal axis. The medial region 126 of the stent 120 may extend between the flared portion 132 of the second end region 124 and the retention member 128 of the first end region 122. The stent 120 may define an open interior lumen (e.g., passage, channel, etc.) extending from the first end region 122 to the second end region 124.

The retention member 128 may extend radially away from (e.g., substantially perpendicular to) the longitudinal axis of the medial region 126 to define a first surface 138a and a second surface 138b. In some instances, the first surface 138a, which may be a distally facing surface of the retention member 128, is substantially parallel to the second surface 138b, which may be a proximally facing surface of the retention member 128. The first surface 138a may be configured to atraumatically engage a (e.g., inner) tissue wall of a first body lumen (e.g., the stomach or duodenum). Further, as will be discussed in greater detail herein, the flared portion 132 (e.g., flared flange structure) of the second end region 124 may include an outer surface 140 configured to atraumatically engage a (e.g., inner) tissue wall of an adjacent or apposed second body lumen (e.g., a biliary duct). In the example stent 120 illustrated in FIG. 2, the surfaces 138a, 140 may prevent or limit movement/migration of the deployed stent 120 within or between the first and second body lumens.

FIG. 2 illustrates that, in some examples, an outer diameter D1 of the retention member 128 may be greater than the outer diameter D2 of the flared portion 132. However, in other examples, the outer diameter D1 of the retention member 128 may be equal to the outer diameter D2 of the flared portion 132. The medial region 126 may include a constant outer diameter D3 extending between the flared portion 132 and the retention member 128, whereby the diameter D3 of the medial region 126 is less than the diameters D1 and D2 of the retention member 128 and the flared portion 132, respectively. As discussed herein, in some examples the outer diameter D3 of the medial region 126 may be substantially equal to the outer diameter D2 of the flared portion 132.

In some examples, the diameter D1 may be about 5 mm to about 40 mm, or about 10 mm to about 35 mm, or about 15 mm to about 30 mm, or about 20 mm to about 25 mm, or about 10 mm to about 20 mm, or about 20 mm to about 35 mm. In some examples, the diameter D2 may be about 2 mm to about 40 mm, or about 6 mm to about 30 mm, or about 10 mm to about 25 mm, or about 15 mm to about 20 mm, or about 6 mm to about 20 mm, or about 15 mm to about 35 mm. In some examples, the diameter D3 may be about 2 mm to about 20 mm, or about 4 mm to about 18 mm, or about 6 mm to about 14 mm, or about 8 mm to about 12 mm, or about 6 mm to about 14 mm, or about 15 mm to about 25 mm.

In one embodiment, the second end region 124 of the stent 120 may include an atraumatic configuration in which free ends of the one or more adjacent woven, braided or knitted strut members 142 are bent and connected to form a series of atraumatic loop ends 144. For example, each loop end 144a may be formed by mating and securing the adjacent free ends of the one or more filaments, e.g., by a weld, solder, adhesive, clamp, crimpable hypotube, or other suitable means as are known in the art. In another embodiment, the second end region 124 of the elongate tubular scaffold of the stent 120 may include an atraumatic configuration (e.g., looped ends) in which the one or more strut members 142 are woven, braided or knitted on a mandrel. Although depicted schematically in FIG. 2, the loop ends 144a may include a variety of substantially angular configurations, including, by way of non-limiting example, semi-circular, semi-elliptical and other smoothly curved or substantially smoothly curved shapes.

Additionally, in some examples, the first end region 122 of the stent 120 may include free ends 146 of the one or more woven, braided or knitted strut members 142 which are not connected, and instead form sharp or pointed free ends of the strut members 142. As will be understood by those of skill in the art, the surface 138a of the retention member 128 may atraumatically engage an inner tissue wall of a first body lumen such that the free ends 146 extend into the first body lumen and do not contact the tissue wall.

As discussed herein, for HGS patients, stent migration may cause serious complications including death. If there is no tissue ingrowth-based adhesion at various anatomical regions, a deployed stent may migrate proximally into the stomach, causing leakage of biliary contents into the peritoneum, resulting in peritonitis. If there is sufficient or excessive adhesion at the hepatic duct, the stent may migrate distally into the peritoneum, causing leakage of biliary and stomach contents into the peritoneum, also causing peritonitis. Additionally, a migrated stent is free to abrade the outer gastric wall and other organs or vessels in the vicinity. Anatomically, stent migration can occur as a result of the hepatic duct being a generally static vessel, whereas the stomach is a highly motile vessel, as discussed herein. Accordingly, one method to reduce stent migration may include exposing bare metal portions of the stent to the tissue of the body lumen. The stent scaffold may then provide a structure that promotes tissue ingrowth (e.g., a hyperplastic response) into the interstices or openings thereof. The tissue ingrowth may anchor the stent in place and reduce the risk of stent migration.

FIG. 2 illustrates that, in some examples, the tubular scaffold of the stent 120 may include one or more non-covered (e.g., bare) portions designed to promote tissue ingrowth (e.g., a hyperplastic response) into the interstices or openings thereof. The non-covered portions may be devoid of a membrane, coating or other covering and thus the interstices of the tubular scaffold in the non-covered portions are open to tissue ingrowth. For example, FIG. 2 illustrates that the stent 120 may include the non-covered portion 134. It can be appreciated that, in some examples, the non-covered portion 134 of the tubular scaffold of the stent 120 may include the flared portion 132 of the stent 120. In other words, the flared portion 132 of the stent 120 shown in FIG. 2 may be bare (e.g., devoid or free of a membrane, coating, etc.) allowing tissue ingrowth through the interstices of the tubular scaffold along the flared portion 132.

Additionally, FIG. 2 illustrates that the stent 120 may include a membrane 150 (e.g., coating, membrane coating, etc.) extending within the lumen of the tubular scaffold of the stent 120. For example, FIG. 2 illustrates that the membrane 150 may extend along a length L of the tubular scaffold of the stent 120. The length L along which the membrane 150 extends within the lumen of the tubular scaffold of the stent 120 may include a portion of the medial region 126 and the first end region 122 (including the retention member 128). As will be discussed in greater detail below, the membrane 150 may extend from a discrete attachment point along the first end region 122 to a discrete attachment point along the medial region 126, whereby the membrane 150 defines a leak-free, passageway (e.g., channel, lumen, tunnel, etc.) which may permit drainage of bodily substances (e.g., bile) from one anatomical organ (e.g., liver) to another anatomical organ (e.g., stomach). In some examples, the membrane 150 be an elastomeric or non-elastomeric material. For example, the membrane 150 may be a polymeric material, such as silicone, polyurethane, UE, PVDF, PTFE, ePTFE, ChronoFlex® or similar biocompatible polymeric formulations.

It can be further appreciated that the stent 120 may be designed to include regions which do not include the membrane 150 and regions which include the membrane 150 in different configurations than that illustrated in FIG. 2. In other words, the length of the non-covered portion 134 and the covered portion L may differ from that illustrated in FIG. 2. For example, the non-covered portion 134 may be longer than that shown in FIG. 2 (e.g., the non-covered portion may extend to a portion or all of the medial region 126). In other examples, more of the stent 120 may include a membrane 150. This would correspond to a lengthening of the length L, whereby the membrane 150 may extend onto the flared portion 132. It can be appreciated that as the proportion of the stent 120 which includes a membrane 150 increases, the proportion of the stent 120 which does not include a membrane decreases, and vice versa.

In some examples, the ratio of the covered portion L to the non-covered portion may be about 9:1 of the covered portion L to the uncovered portion 134, or about 4:1 of the covered portion L to the uncovered portion 134, or about 7:3 of the covered portion L to the uncovered portion 134, or about 3:2 of the covered portion L to the uncovered portion 134, or about 1:1 of the covered portion L to the uncovered portion 134. The uncovered portion 134 may be designed to be more flexible than the uncovered portion 134, allowing for better response when positioned in highly motile regions of the body. It can be appreciated that designing the stent 120 to include a relatively greater length of the uncovered portion 134 to the covered portion L may dedicate a greater percentage of the overall stent length to bare region duct drainage and anti-migration features. However, while longer uncovered portions 134 may allow for higher potential for side branch drainage and anti-migration ingrowth, the reduction in the covered portion L may reduce the effective length of the stent 120 that can be bridged between disconnected anatomical vessels (e.g., the distance between the gastric wall into the liver parenchyma). The uncovered portion 134 of the stent 120 must be sized to allow enough resistance to stent migration (so as to prevent migration initially through mechanical resistance and eventually with the addition of tissue ingrowth) while also allowing the stent 120 to include enough of a covered portion L to bridge the distance between disconnected anatomical vessels (e.g., the distance between the gastric wall into the liver parenchyma).

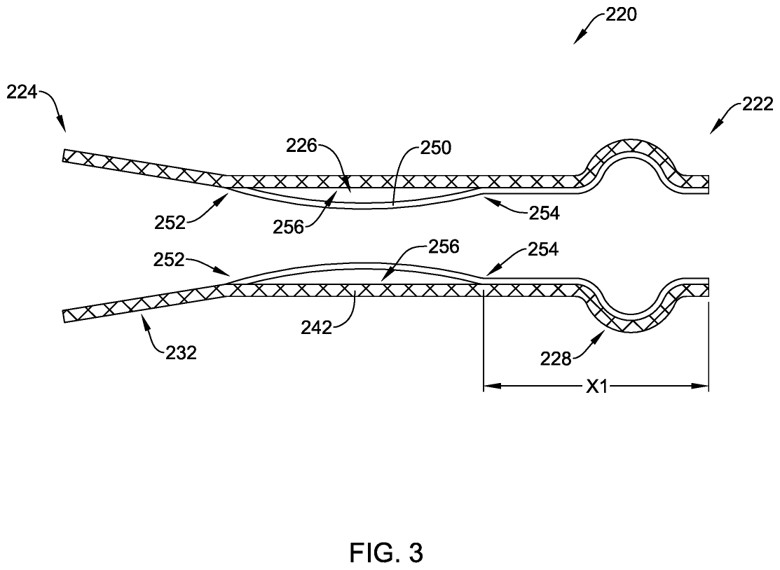

FIGS. 3-9 illustrate example stents that may be similar in form and function to the stent 120 described above. For example, each of the stents shown in FIGS. 3-9 may include a membrane disposed within the lumen of the tubular scaffold of the stent (e.g., as described with respect to FIG. 2). The stents illustrated in FIGS. 3-9 may include various portions of the tubular scaffold of the stent which are uncovered to promote tissue ingrowth therethrough.

FIG. 3 illustrates a cross-sectional view of an example expandable medical device, namely a stent 220. The example stent 220 may be similar in form and function to the stent 120 described herein. For example, the stent 220 may include a first end region 222, a second end region 224 and a medial region 226. The stent 220 may be formed from one or more braided, knitted, wrapped, intertwined, interwoven, weaved, looped (e.g., bobbinet-style) strut members 242 to form the tubular scaffold of the stent 220. The stent 220 may also include a flared portion 232 positioned along the second end region 224. Additionally, the stent may include a retention member 228 positioned along the first end region 222.

FIG. 3 further illustrates that the stent 220 may include a membrane 250 extending along a portion of the inner surface of the strut members 242 defining the tubular scaffold of the stent 220. In other words, FIG. 3 illustrates that the stent 220 may include a membrane 250 extending within the inner lumen of the tubular scaffold of the stent 220, whereby the membrane 250 may be attached at various positions along the inner surface of the tubular scaffold of the stent 220.

For example, FIG. 3 illustrates that membrane 250 may be attached to the tubular scaffold of the stent 220 at a first discrete circumferential attachment point 252 and a second discrete circumferential attachment point 254. It can be appreciated that the membrane 250 may be attached around the entire circumference along the inner surface of the tubular scaffold of the stent 220 at both the first discrete circumferential attachment point 252 and the second discrete circumferential attachment point 254. It can be further appreciated from FIG. 3 that the membrane 250 may extend away from the inner surface 256 of the strut members 242 (which form the tubular scaffold of the stent 220) along the portion of the stent structure which extends between the first discrete circumferential attachment point 252 and the second discrete circumferential attachment point 254.

Additionally, FIG. 3 illustrates that membrane 250 may be fixedly attached to the inner surface of the tubular scaffold of the stent 220 along a portion of the tubular scaffold of the stent 220 identified as a length X1. FIG. 3 shows that membrane 250 may be adhered (e.g., affixed, secured, etc.) to the inner surface of strut members 242 along the length X1. It can be appreciated from FIG. 3 that the length X1 of the tubular scaffold of the stent 220 may include the retention member 228. It can be appreciated that the portions of tubular scaffold of the stent 220 discussed above which include the membrane 250 which is attached (e.g., covers) to the strut members 242 may act to prevent tissue from growing into the interstices or openings thereof. For example, the strut members 242 along the length X1 of tubular scaffold of the stent 220 which include the membrane 250 attached thereto may span across interstices of the tubular scaffold of the stent 220 and may prevent tissue ingrowth along their respective surfaces and interstices therebetween.

Further, it can be appreciated that tissue may be permitted to grow around, between, through, within, etc. those strut members 242 of the tubular scaffold of the stent 220 in which the membrane 250 is not attached (e.g., the portion of membrane 250 extending along the flared portion 232 and the portion of the membrane 250 extending along the medial region 226 between the first discrete circumferential attachment point 252 and the second discrete circumferential attachment point 254 of the tubular scaffold of the stent 220). In other words, FIG. 3 illustrates multiple “tissue ingrowth regions” defined along both the flared portion 232 and along the medial region 226 (between the first discrete circumferential attachment point 252 and the second discrete circumferential attachment point 254) of the tubular scaffold of the stent 220. The tissue ingrowth regions may be defined as the space between the inner surface 256 of the tubular scaffold of the stent 220 and the outer surface of the membrane 250 extending between attachment points 252/254. As discussed herein, uncovered or bare portions of the tubular scaffold of the stent 220 may provide structures that promote tissue ingrowth to anchor the stent 220 in place and reduce the risk of stent migration.

Further, it can be appreciated from the discussion herein regarding the discrete attachment points of the membrane 250 to the inner surface of the tubular scaffold of the stent 220 that the membrane 250 may define a leak-free, passageway (e.g., channel, lumen, tunnel, etc.) which may permit drainage of bodily substances (e.g., bile) from one anatomical organ (e.g., liver) to another anatomical organ (e.g., stomach).

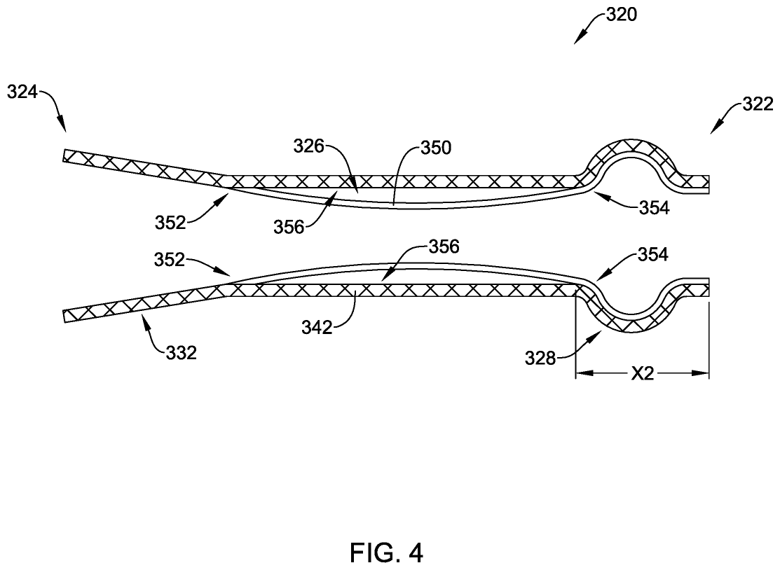

FIG. 4 illustrates a cross-sectional view of another example expandable medical device, namely a stent 320. The example stent 320 may be similar in form and function to the stent 120 described herein. For example, the stent 320 may include a first end region 322, a second end region 324 and a medial region 326. The stent 320 may be formed from one or more braided, knitted, wrapped, intertwined, interwoven, weaved, looped (e.g., bobbinet-style) strut members 342 to form the tubular scaffold of the stent 320. The stent 320 may also include a flared portion 332 positioned along the second end region 324. Additionally, the stent may include a retention member 328 positioned along the first end region 322.

FIG. 4 further illustrates that the stent 320 may include a membrane 350 extending along a portion of the inner surface of the strut members 342 defining the tubular scaffold of the stent 320. In other words, FIG. 4 illustrates that the stent 320 may include a membrane 350 extending within the inner lumen of the tubular scaffold of the stent 320, whereby the membrane 350 may be attached at various positions along the inner surface of the tubular scaffold of the stent 320.

For example, FIG. 4 illustrates that membrane 350 may be attached to the tubular scaffold of the stent 320 at a first discrete circumferential attachment point 352 and a second discrete circumferential attachment point 354. It can be appreciated that the membrane 350 may be attached around the entire circumference along the inner surface of the tubular scaffold of the stent 320 at both the first discrete circumferential attachment point 352 and the second discrete circumferential attachment point 354. It can be further appreciated from FIG. 4 that the membrane 350 may extend away from the inner surface 356 of the strut members 342 (which form the tubular scaffold of the stent 320) along the portion of the stent structure which extends between the first discrete circumferential attachment point 352 and the second discrete circumferential attachment point 354.

Additionally, FIG. 4 illustrates that membrane 350 may be fixedly attached to the inner surface of the tubular scaffold of the stent 320 along a portion of the tubular scaffold of the stent 320 identified as a length X2. FIG. 4 shows that membrane 350 may be adhered (e.g., affixed, secured, etc.) to the inner surface of strut members 342 along the length X2. It can be appreciated from FIG. 4 that the length X2 of the tubular scaffold of the stent 320 may include the retention member 328. It can be appreciated that the portions of tubular scaffold of the stent 320 discussed above which include the membrane 350 which is attached (e.g., covers) to the strut members 342 may act to prevent tissue from growing into the interstices or openings thereof. For example, the strut members 342 along the length X2 of tubular scaffold of the stent 320 which include the membrane 350 attached thereto may span across interstices of the tubular scaffold of the stent 320 and may prevent tissue ingrowth along their respective surfaces and interstices therebetween.

Further, it can be appreciated that tissue may be permitted to grow around, between, through, within, etc. those strut members 342 of the tubular scaffold of the stent 320 in which the membrane 350 is not attached (e.g., the portion of membrane 350 extending along the flared portion 332 and the portion of the membrane 350 extending along the medial region 326 between the first discrete circumferential attachment point 352 and the second discrete circumferential attachment point 354 of the tubular scaffold of the stent 320). In other words, FIG. 4 illustrates multiple “tissue ingrowth regions” defined along both the flared portion 332 and along the medial region 326 (between the first discrete circumferential attachment point 352 and the second discrete circumferential attachment point 354) of the tubular scaffold of the stent 320. The tissue ingrowth regions may be defined as the space between the inner surface 356 of the tubular scaffold of the stent 320 and the outer surface of the membrane 350 extending between discrete attachment points 352/354. As discussed herein, uncovered or bare portions of the tubular scaffold of the stent 320 may provide structures that promote tissue ingrowth to anchor the stent 320 in place and reduce the risk of stent migration.

Further, it can be appreciated from the discussion herein regarding the discrete attachment points of the membrane 350 to the inner surface of the tubular scaffold of the stent 320 that the membrane 350 may define a leak-free, passageway (e.g., channel, lumen, tunnel, etc.) which may permit drainage of bodily substances (e.g., bile) from one anatomical organ (e.g., liver) to another anatomical organ (e.g., stomach).

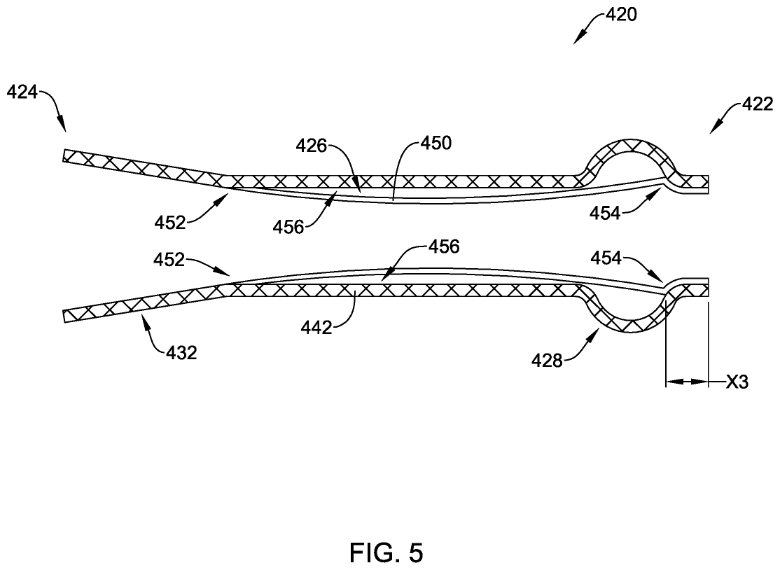

FIG. 5 illustrates a cross-sectional view of another example expandable medical device, namely a stent 420. The example stent 420 may be similar in form and function to the stent 120 described herein. For example, the stent 420 may include a first end region 422, a second end region 424 and a medial region 426. The stent 420 may be formed from one or more braided, knitted, wrapped, intertwined, interwoven, weaved, looped (e.g., bobbinet-style) strut members 442 to form the tubular scaffold of the stent 420. The stent 420 may also include a flared portion 432 positioned along the second end region 424. Additionally, the stent may include a retention member 428 positioned along the first end region 422.

FIG. 5 further illustrates that the stent 420 may include a membrane 450 extending along a portion of the inner surface of the strut members 442 defining the tubular scaffold of the stent 420. In other words, FIG. 5 illustrates that the stent 420 may include a membrane 450 extending within the inner lumen of the tubular scaffold of the stent 420, whereby the membrane 450 may be attached at various positions along the inner surface of the tubular scaffold of the stent 420.

For example, FIG. 5 illustrates that membrane 450 may be attached to the tubular scaffold of the stent 420 at a first discrete circumferential attachment point 452 and a second discrete circumferential attachment point 454. It can be appreciated that the membrane 450 may be attached around the entire circumference along the inner surface of the tubular scaffold of the stent 420 at both the first discrete circumferential attachment point 452 and the second discrete circumferential attachment point 454. It can be further appreciated from FIG. 5 that the membrane 450 may extend away from the inner surface 456 of the strut members 442 (which form the tubular scaffold of the stent 420) along the portion of the stent structure which extends between the first discrete circumferential attachment point 452 and the second discrete circumferential attachment point 454.

Additionally, FIG. 5 illustrates that membrane 450 may be fixedly attached to the inner surface of the tubular scaffold of the stent 420 along a portion of the tubular scaffold of the stent 420 identified as a length X3. FIG. 5 shows that membrane 450 may be adhered (e.g., affixed, secured, etc.) to the inner surface of strut members 442 along the length X3. It can be appreciated from FIG. 4 that the length X3 of the tubular scaffold of the stent 420 may include the retention member 428. It can be appreciated that the portions of tubular scaffold of the stent 420 discussed above which include the membrane 450 which is attached (e.g., covers) to the strut members 442 may act to prevent tissue from growing into the interstices or openings thereof. For example, the strut members 442 along the length X3 of tubular scaffold of the stent 420 which include the membrane 450 attached thereto may span across interstices of the tubular scaffold of the stent 420 and may prevent tissue ingrowth along their respective surfaces and interstices therebetween.

Further, it can be appreciated that tissue may be permitted to grow around, between, through, within, etc. those strut members 442 of the tubular scaffold of the stent 420 in which the membrane 450 is not attached (e.g., the portion of membrane 450 extending along the flared portion 432 and the portion of the membrane 450 extending along the medial region 426 between the first discrete circumferential attachment point 452 and the second discrete circumferential attachment point 454 of the tubular scaffold of the stent 420). In other words, FIG. 5 illustrates multiple “tissue ingrowth regions” defined along both the flared portion 432 and along the medial region 426 (between the first discrete circumferential attachment point 452 and the second discrete circumferential attachment point 454) of the tubular scaffold of the stent 420. The tissue ingrowth regions may be defined as the space between the inner surface 456 of the tubular scaffold of the stent 420 and the outer surface of the membrane 450 extending between discrete attachment points 452/454. As discussed herein, uncovered or bare portions of the tubular scaffold of the stent 420 may provide structures that promote tissue ingrowth to anchor the stent 420 in place and reduce the risk of stent migration.

Further, it can be appreciated from the discussion herein regarding the discrete attachment points of the membrane 450 to the inner surface of the tubular scaffold of the stent 420 that the membrane 450 may define a leak-free, passageway (e.g., channel, lumen, tunnel, etc.) which may permit drainage of bodily substances (e.g., bile) from one anatomical organ (e.g., liver) to another anatomical organ (e.g., stomach).

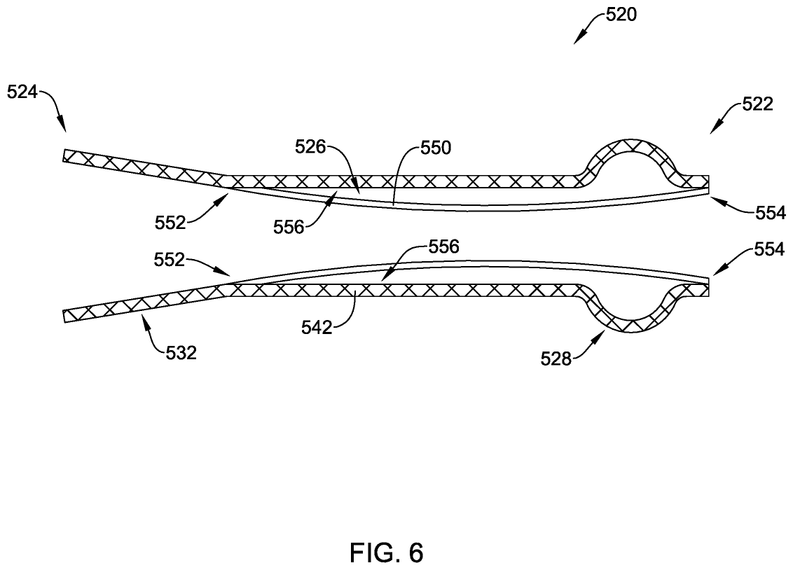

FIG. 6 illustrates a cross-sectional view of another example expandable medical device, namely a stent 520. The example stent 520 may be similar in form and function to the stent 120 described herein. For example, the stent 520 may include a first end region 522, a second end region 524 and a medial region 526. The stent 520 may be formed from one or more braided, knitted, wrapped, intertwined, interwoven, weaved, looped (e.g., bobbinet-style) strut members 542 to form the tubular scaffold of the stent 520. The stent 520 may also include a flared portion 532 positioned along the second end region 524. Additionally, the stent may include a retention member 528 positioned along the first end region 522.

FIG. 6 further illustrates that the stent 520 may include a membrane 550 extending along a portion of the inner surface of the strut members 542 defining the tubular scaffold of the stent 520. In other words, FIG. 6 illustrates that the stent 520 may include a membrane 550 extending within the inner lumen of the tubular scaffold of the stent 520, whereby the membrane 550 may be attached at various positions along the inner surface of the tubular scaffold of the stent 520.

For example, FIG. 6 illustrates that membrane 550 may be attached to the tubular scaffold of the stent 520 at a first discrete circumferential attachment point 552 and a second discrete circumferential attachment point 554. It can be appreciated that the membrane 550 may be attached around the entire circumference along the inner surface of the tubular scaffold of the stent 520 at both the first discrete circumferential attachment point 552 and the second discrete circumferential attachment point 554. It can be further appreciated from FIG. 6 that the membrane 550 may extend away from the inner surface 556 of the strut members 542 (which form the tubular scaffold of the stent 520) along the portion of the stent structure which extends between the first discrete circumferential attachment point 552 and the second discrete circumferential attachment point 554.

It can be appreciated that the portions of tubular scaffold of the stent 520 discussed above which include the membrane 550 which is attached (e.g., covers) to the strut members 542 may act to prevent tissue from growing into the interstices or openings thereof. For example, the strut members 542 of tubular scaffold of the stent 520 which include the membrane 550 attached thereto may span across interstices of the tubular scaffold of the stent 520 and may prevent tissue ingrowth along their respective surfaces and interstices therebetween.

Further, it can be appreciated that tissue may be permitted to grow around, between, through, within, etc. those strut members 542 of the tubular scaffold of the stent 520 in which the membrane 550 is not attached (e.g., the portion of membrane 550 extending along the flared portion 532 and the portion of the membrane 550 extending along the medial region 526 between the first discrete circumferential attachment point 552 and the second discrete circumferential attachment point 554 of the tubular scaffold of the stent 520). In other words, FIG. 6 illustrates multiple “tissue ingrowth regions” defined along both the flared portion 532 and along the medial region 526 (between the first discrete circumferential attachment point 552 and the second discrete circumferential attachment point 554) of the tubular scaffold of the stent 520. The tissue ingrowth regions may be defined as the space between the inner surface 556 of the tubular scaffold of the stent 520 and the outer surface of the membrane 550 extending between discrete attachment points 552/554. As discussed herein, uncovered or bare portions of the tubular scaffold of the stent 520 may provide structures that promote tissue ingrowth to anchor the stent 520 in place and reduce the risk of stent migration.

Further, it can be appreciated from the discussion herein regarding the discrete attachment points of the membrane 550 to the inner surface of the tubular scaffold of the stent 520 that the membrane 550 may define a leak-free, passageway (e.g., channel, lumen, tunnel, etc.) which may permit drainage of bodily substances (e.g., bile) from one anatomical organ (e.g., liver) to another anatomical organ (e.g., stomach).

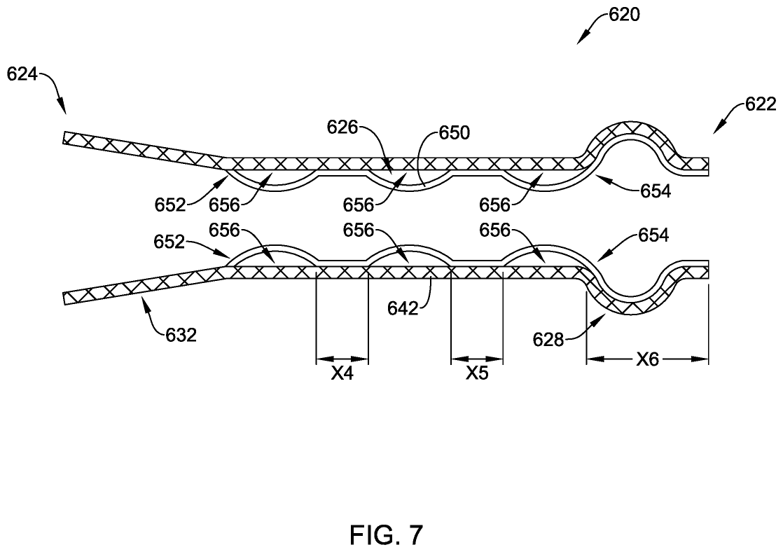

FIG. 7 illustrates a cross-sectional view of another example expandable medical device, namely a stent 620. The example stent 620 may be similar in form and function to the stent 620 described herein. For example, the stent 620 may include a first end region 622, a second end region 624 and a medial region 626. The stent 620 may be formed from one or more braided, knitted, wrapped, intertwined, interwoven, weaved, looped (e.g., bobbinet-style) strut members 642 to form the tubular scaffold of the stent 620. The stent 620 may also include a flared portion 632 positioned along the second end region 624. Additionally, the stent may include a retention member 628 positioned along the first end region 622.

FIG. 7 further illustrates that the stent 620 may include a membrane 650 extending along a portion of the inner surface of the strut members 642 defining the tubular scaffold of the stent 620. In other words, FIG. 7 illustrates that the stent 620 may include a membrane 650 extending within the inner lumen of the tubular scaffold of the stent 620, whereby the membrane 650 may be attached at various positions along the inner surface of the tubular scaffold of the stent 620.

For example, FIG. 7 illustrates that the membrane 650 may be attached to the tubular scaffold of the stent 620 at a first discrete circumferential attachment point 652 and a second discrete circumferential attachment point 654. It can be appreciated that the membrane 650 may be attached around the entire circumference along the inner surface of the tubular scaffold of the stent 620 at both the first discrete circumferential attachment point 652 and the second discrete circumferential attachment point 654.

Additionally, FIG. 7 illustrates that membrane 650 may be fixedly attached to the inner surface of the tubular scaffold of the stent 620 along portions of the tubular scaffold of the stent 620 identified as lengths X4, X5, X6. The length X6 for which the that membrane 650 may be fixedly attached to the inner surface of the tubular scaffold of the stent 620 may be similar to the length X2 described with respect to FIG. 4. FIG. 7 shows that membrane 650 may be adhered (e.g., affixed, secured, etc.) to the inner surface of strut members 642 along the lengths X4, X5, X6 of the tubular scaffold of the stent 620. It can be appreciated from FIG. 7 that the length X6 of the tubular scaffold of the stent 620 may include the retention member 628. It can be appreciated that the portions of tubular scaffold of the stent 620 discussed above which include the membrane 650 which is attached (e.g., covers) to the strut members 642 may act to prevent tissue from growing into the interstices or openings thereof. For example, the strut members 642 along the lengths X4, X5, X6 of tubular scaffold of the stent 620 which include the membrane 650 attached thereto may span across interstices of the tubular scaffold of the stent 620 and may prevent tissue ingrowth along their respective surfaces and interstices therebetween.

Further, it can be appreciated that tissue may be permitted to grow around, between, through, within, etc. those strut members 642 of the tubular scaffold of the stent 620 in which the membrane 650 is not attached (e.g., the portion of membrane 650 extending along the flared portion 632 and the portion of the membrane 650 extending along the medial region 626 between the first discrete circumferential attachment point 352 and the length X4, between the length X4 and the length X5, between the length X5 and the length X6, and along the length X6). In other words, FIG. 7 illustrates multiple “tissue ingrowth regions” defined along both the flared portion 632 and along the medial region 626 (between the first discrete circumferential attachment point 652 and the length X4, between the length X4 and the length X5 and between the length X5 and the length X6) of the tubular scaffold of the stent 620. The tissue ingrowth regions may be defined as the space between the inner surface 656 of the tubular scaffold of the stent 620 and the outer surface of the membrane 650 extending between discrete attachment points 652/654. As discussed herein, uncovered or bare portions of the tubular scaffold of the stent 620 may provide structures that promote tissue ingrowth to anchor the stent 620 in place and reduce the risk of stent migration.

It can be appreciated that in some examples the length X4 may be substantially equal to the length X5. However, in other examples, the length X4 may be different than the length X5. For example, the length X4 may be shorter than the length X5. In other examples, the length X5 may be shorter than the length X4. It can be further appreciated that the distance from which the membrane 650 extends away from the inner surface 656 of the stent 620 may vary depending on the length of X4 or X5. For example, it can be appreciated that decreasing the length X4 or X5 may result in a lengthening of the unattached portions of the membrane 650 extending between the portions of the membrane 650 which remain attached to the inner surface 656 of the stent 620. Longer unattached portions of the membrane 650 may permit the membrane 650 to extend farther away from the inner surface 656, thereby resulting in larger ingrowth “pockets” that allow a greater volume of tissue ingrowth to anchor the stent 120.

Further, it can be appreciated from the discussion herein regarding the discrete attachment points of the membrane 650 to the inner surface 656 of the tubular scaffold of the stent 620 that the membrane 650 may define a leak-free, passageway (e.g., channel, lumen, tunnel, etc.) which may permit drainage of bodily substances (e.g., bile) from one anatomical organ (e.g., liver) to another anatomical organ (e.g., stomach).

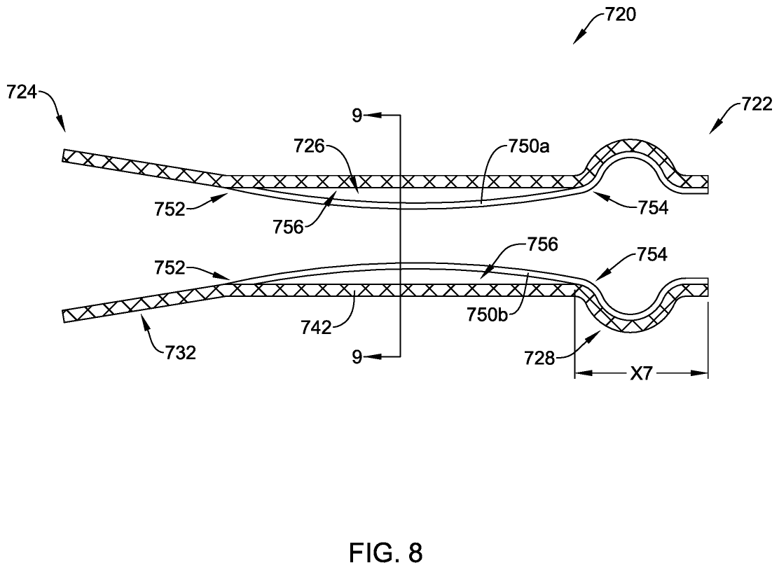

FIG. 8 illustrates a cross-sectional view of another example expandable medical device, namely a stent 720. The example stent 720 may be similar in form and function to the stent 120 described herein. For example, the stent 720 may include a first end region 722, a second end region 724 and a medial region 726. The stent 720 may be formed from one or more braided, knitted, wrapped, intertwined, interwoven, weaved, looped (e.g., bobbinet-style) strut members 742 to form the tubular scaffold of the stent 720. The stent 720 may also include a flared portion 732 positioned along the second end region 724. Additionally, the stent may include a retention member 728 positioned along the first end region 722.

FIG. 8 further illustrates that the stent 720 may include a membrane 750 extending along a portion of the inner surface of the strut members 742 defining the tubular scaffold of the stent 720. In other words, FIG. 8 illustrates that the stent 720 may include a membrane 750 extending within the inner lumen of the tubular scaffold of the stent 720, whereby the membrane 750 may be attached at various positions along the inner surface of the tubular scaffold of the stent 720.

For example, FIG. 8 illustrates that membrane 750 may be attached to the tubular scaffold of the stent 720 at a first discrete circumferential attachment point 752 and a second discrete circumferential attachment point 754. It can be appreciated that the membrane 750 may be attached around the entire circumference along the inner surface of the tubular scaffold of the stent 720 at both the first discrete circumferential attachment point 752 and the second discrete circumferential attachment point 754.

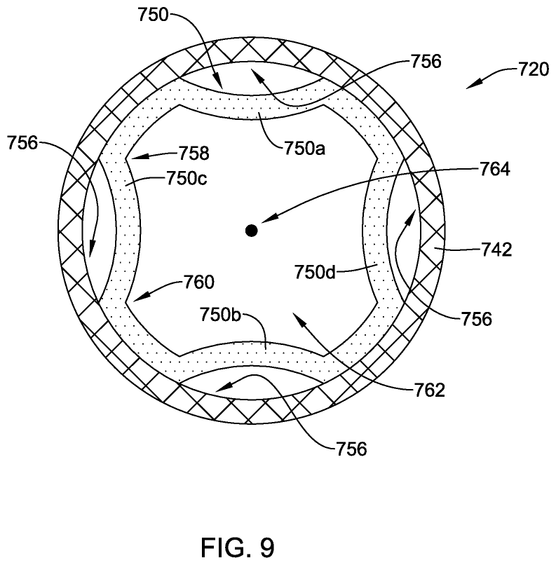

Further, FIG. 8 illustrates an example in which the membrane 750 is attached and unattached to the tubular scaffold of the stent 720 in a longitudinally striped configuration. The striped configuration of the unattached portions of the membrane 750 is further illustrated in the cross-sectional view taken along line 9-9 of FIG. 8. For example, the cross-sectional view of FIG. 9 illustrates four portions 750a, 750b, 750c, 750d of the membrane 750 which are unattached to the tubular scaffold of the stent 720. It can be appreciated from FIG. 9 that each of the four portions 750a, 750b, 750c, 750d of the membrane 750 which are unattached to the tubular scaffold of the stent 720 extend inward toward the longitudinal axis 764 of the stent 720. FIG. 9 illustrates that each of the four portions 750a, 750b, 750c, 750d of the membrane 750 create a space between the inner surface of the stent 720 and an outwardly-facing surface of the membrane 750. Further, the cross-sectional view of FIG. 9 illustrates that the membrane 750 may be connected to the inner surface 756 of the tubular scaffold of the stent 720 along the portions of the stent extending between the four portions 750a, 750b, 750c. 750d of the membrane 750 which are unattached to the tubular scaffold of the stent 720. In other words, FIG. 9 illustrates that the membrane 750 may be circumferentially continuous such that it defines a lumen 762 extending longitudinally along the tubular scaffold of the stent 720. It can be further appreciated from FIG. 8 that the membrane 750 may extend away from the inner surface 756 of the strut members 742 (which form the tubular scaffold of the stent 720) along the portion of the stent structure which extends between the first discrete circumferential attachment point 752 and the second discrete circumferential attachment point 754.

Additionally, FIG. 9 illustrates that the membrane 750 may be connected to the inner surface 756 of the tubular scaffold of the stent 720 along the portions of the stent between the four portions 750a, 750b, 750c, 750d of the membrane 750 which are unattached to the tubular scaffold of the stent 720. Further, FIG. 8 illustrates that membrane 750 may be attached along the portion of the tubular scaffold of the stent 320 identified as a length X7. FIG. 9 shows that membrane 750 may be adhered (e.g., affixed, secured, etc.) to the inner surface of strut members 742 along the length X7. It can be appreciated from FIG. 8 that the length X7 of the tubular scaffold of the stent 720 may include the retention member 728. It can be appreciated that the portions of tubular scaffold of the stent 720 discussed above which include the membrane 750 which is attached (e.g., covers) to the strut members 742 may act to prevent tissue from growing into the interstices or openings thereof. For example, the strut members 742 along the length X7 of tubular scaffold of the stent 720 which include the membrane 750 attached thereto may span across interstices of the tubular scaffold of the stent 720 and may prevent tissue ingrowth along their respective surfaces and interstices therebetween.

Further, it can be appreciated that tissue may be permitted to grow around, between, through, within, etc. those strut members 742 of the tubular scaffold of the stent 720 in which the membrane 750 is not attached (e.g., the portion of membrane 750 extending along the flared portion 732 and the portion of the membrane 750 extending between the four portions 750a. 750b, 750c, 750d of the membrane 750 which are unattached to the tubular scaffold of the stent 720). In other words, FIGS. 8-9 illustrate multiple “tissue ingrowth regions” defined along both the flared portion 732 and along the medial region 726 (between the four portions 750a, 750b, 750c, 750d of the membrane 750 which are unattached to the tubular scaffold of the stent 720) of the tubular scaffold of the stent 720. The tissue ingrowth regions may be defined as the space between the inner surface 756 of the tubular scaffold of the stent 720 and the outer surface of the membrane 750. As discussed herein, uncovered or bare portions of the tubular scaffold of the stent 720 may provide structures that promote tissue ingrowth to anchor the stent 720 in place and reduce the risk of stent migration.

Further, it can be appreciated from the discussion herein regarding the discrete attachment points of the membrane 750 to the inner surface of the tubular scaffold of the stent 720 that the membrane 750 may define a leak-free, passageway (e.g., channel, lumen, tunnel, etc.) which may permit drainage of bodily substances (e.g., bile) from one anatomical organ (e.g., liver) to another anatomical organ (e.g., stomach).

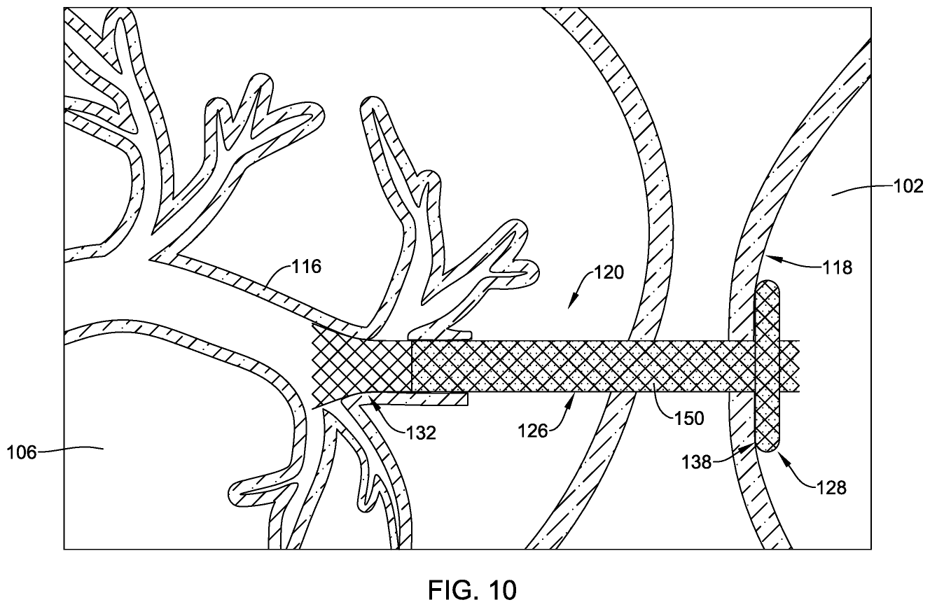

As discussed herein, the examples stents described herein may be designed to permit leak-free drainage from one anatomical structure (e.g., hepatic ducts) to another anatomical structure (e.g., stomach) while also permitting tissue ingrowth into the stent to prevent stent migration. For example, FIG. 10 illustrates the flared end portion 132 of the example stent 120 positioned in a hepatic duct 116 of the liver 106. FIG. 10 also illustrates the retention member 128 positioned in the stomach 102.

As discussed herein, the uncovered portions of the stent 120 may encourage tissue ingrowth, which can be desirable in order to prevent migration of stent 120 after it has been appropriately positioned within the body. For example, configuration of the example stents disclosed herein may allow for resistance to migration based on the selective allowance of tissue ingrowth along uncovered portions of the stent. For example, the atraumatic surface 138a (shown contacting the inner wall 118 of the stomach 102) of the retention member 128 and also along the flared portion 132 of the stent 120 may allow for resistance to migration based on the selective allowance of tissue ingrowth along those portions. As described herein, other stent designs disclosed herein may include additional regions for the allowance of tissue ingrowth. Additionally, FIG. 10 illustrates that the stent 120 may permit the flow of bile (or other body fluid) from the haptic duct 116 to the stomach 102 via the lumen of the membrane 150.

The example stents shown in FIGS. 2-9 may contemplate braided stent designs which are utilized to permit leak-free drainage from one anatomical structure (e.g., hepatic ducts) to another anatomical structure (e.g., stomach) while also permitting tissue ingrowth into the stent to prevent stent migration. Additional stent designs are contemplated to permit leak-free drainage from one anatomical structure (e.g., hepatic ducts) to another anatomical structure (e.g., stomach) while also permitting tissue ingrowth into the stent to prevent stent migration. For example, knitted stents described herein may be utilized for the same purposes as the stents described in FIGS. 2-9.

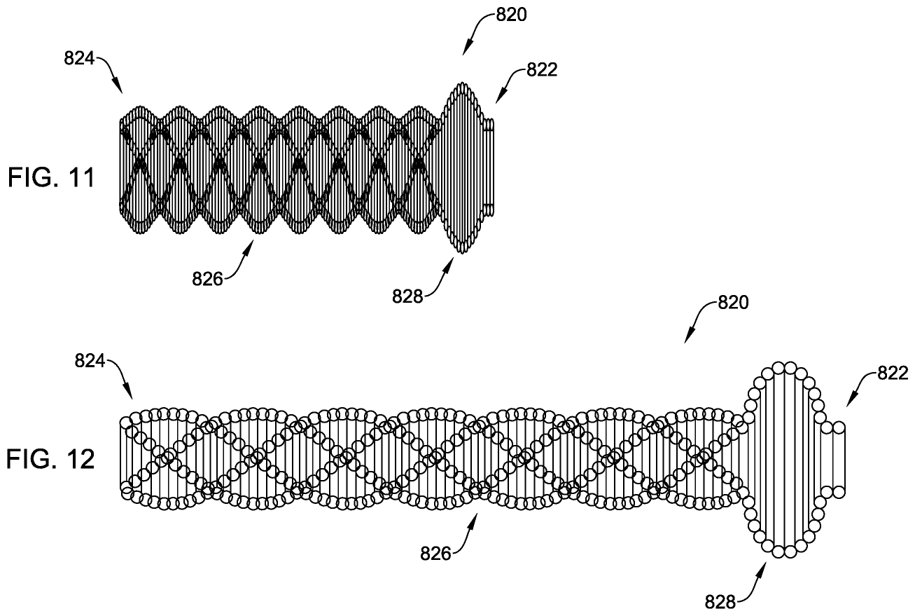

FIG. 11 illustrates an example expandable medical device, namely a knitted stent 820. The stent 820 may have a first end 822, a second end 824 and a medial region 826 extending between the first end 822 and the second end 824. The stent 820 may include a lumen extending from a first opening adjacent the first end 822 to a second opening adjacent to the second end 824. The stent 820 may be fabricated from at least one filament defining open cells and twisted knit stitches. In some examples, the stent 820 may be formed from only a single filament interwoven with itself to form open cells and twisted knit stitches. In some cases, the filament may be a monofilament, while in other cases the filament may be two or more filaments wound, braided, or woven together.

It is contemplated that the stent 820 may be made from a number of different materials such as, but not limited to, metals, metal alloys, shape memory alloys and/or polymers, as desired, enabling the stent 820 to be expanded into shape when accurately positioned within the body. In some instances, the material may be selected to enable the stent 820 to be removed with relative ease as well. For example, the stent 820 can be formed from alloys such as, but not limited to, Nitinol and Elgiloy®. Depending on the material selected for construction, the stent 820 may be self-expanding (i.e., configured to automatically radially expand when unconstrained). In some embodiments, fibers may be used to make the stent 820, which may be composite fibers, for example, having an outer shell made of Nitinol having a platinum core. It is further contemplated the stent 820 may be formed from polymers including, but not limited to, polyethylene terephthalate (PET).

Elongation of the disclosed knitted pattern, as shown in FIG. 11, may permit the stent 820 to change in length without a significant reduction in diameter, and also to maintain radial force as the excess material ‘stored’ in the design is available, effectively over the span of the design as an ‘infinite’ braid. FIGS. 11-12 demonstrate the performance of the knitted stent 820 as it moves between a relaxed, longitudinally contracted state (shown in FIG. 11) to an elongated state (shown in FIG. 12). The knitted stent 820 may accommodate a length change without a change in diameter, as shown in FIGS. 11-12. The stent 820 may thus have a first longitudinal length and a first diameter in the relaxed, expanded configuration (FIG. 11) and a second longitudinal length and a second diameter in the elongated configuration (FIG. 12), where the first longitudinal length is less than the second longitudinal length, and the first and second diameters may be substantially the same. As a result, the stent 820 may be able to compensate for more movement between two anatomical structures, and may perform better in motile areas such as for the HGS procedure described above, while maintaining a substantially constant lumen diameter allowing for fluid flow through the stent. The stent 820 may behave like a spring in the deployed, relaxed and fully expanded configuration.

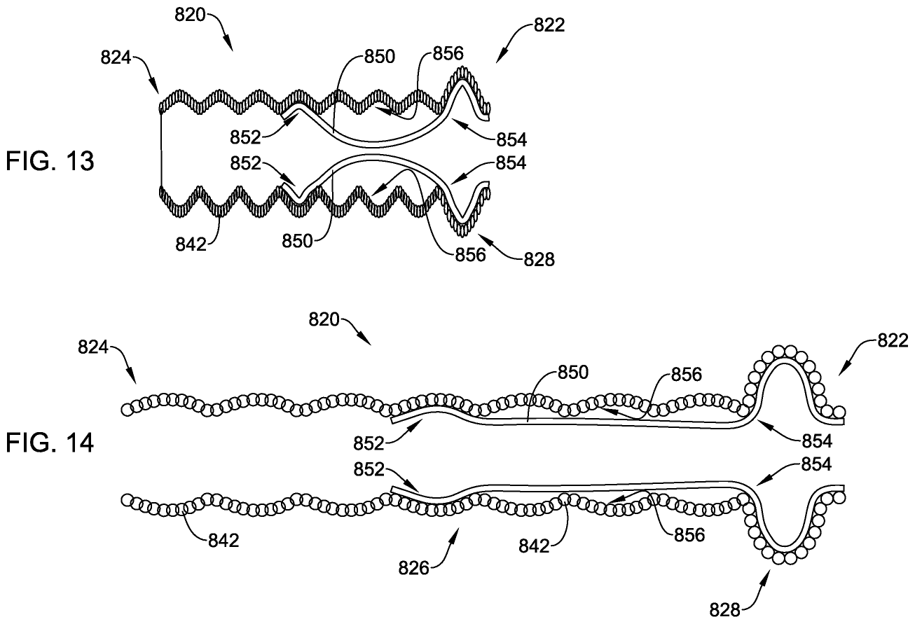

FIG. 13 illustrates a cross-sectional view of the stent 820 shown in FIG. 11. FIG. 13 further illustrates that the stent 820 may include a membrane 850 extending along a portion of the inner surface of the knitted filament members 842 defining the knitted scaffold of the stent 820. In other words, FIG. 13 illustrates that the stent 820 may include a membrane 850 extending within the inner lumen of the knitted scaffold of the stent 820, whereby the membrane 850 may be attached at various positions along the inner surface of the knitted scaffold of the stent 820.

For example, FIG. 13 illustrates that membrane 850 may be attached to the knitted scaffold of the stent 820 at a first discrete circumferential attachment position 852 and a second discrete circumferential attachment position 854. It can be appreciated that the membrane 850 may be attached around the entire circumference along the inner surface of the knitted scaffold of the stent 820 at both the first discrete circumferential attachment position 852 and the second discrete circumferential attachment position 854. It can be further appreciated from FIG. 13 that the membrane 850 may extend away from the inner surface 856 of the knitted filament members 842 (which form the knitted scaffold of the stent 820) along the portion of the stent structure which extends between the first discrete circumferential attachment position 852 and the second discrete circumferential attachment position 854.

Additionally, FIG. 13 illustrates that membrane 850 may be fixedly attached to the inner surface of the tubular scaffold of the stent 820 along the retention member 828 of the knitted scaffold of the stent 820. FIG. 13 shows that membrane 850 may be adhered (e.g., affixed, secured, etc.) to the inner surface of knitted filament members 842 along the retention member 828. It can be appreciated that the portions of knitted scaffold of the stent 820 discussed above which include the membrane 850 which is attached (e.g., covers) to the knitted filament members 842 may act to prevent tissue from growing into the interstices or openings thereof. For example, the knitted filament members 842 along the retention member 828 of knitted scaffold of the stent 820 which include the membrane 850 attached thereto may span across interstices of the knitted scaffold of the stent 820 and may prevent tissue ingrowth along their respective surfaces and interstices therebetween.

Further, it can be appreciated that tissue may be permitted to grow around, between, through, within, etc. those knitted filament members 842 of the tubular scaffold of the stent 820 in which the membrane 850 is not attached. In other words, FIG. 13 illustrates multiple “tissue ingrowth regions” defined along the medial region 826 of the knitted scaffold of the stent 820. Some tissue ingrowth regions may be defined as the space between the inner surface 856 of the tubular scaffold of the stent 820 and the outer surface of the membrane 850 extending between discrete attachment points 852/854. As discussed herein, uncovered or bare portions of the knitted scaffold of the stent 820 may provide structures that promote tissue ingrowth to anchor the stent 820 in place and reduce the risk of stent migration.

Further, it can be appreciated from the discussion herein regarding the discrete attachment points of the membrane 850 to the inner surface of the knitted scaffold of the stent 820 that the membrane 850 may define a leak-free, passageway (e.g., channel, lumen, tunnel, etc.) which may permit drainage of bodily substances (e.g., bile) from one anatomical organ (e.g., liver) to another anatomical organ (e.g., stomach).

FIG. 14 illustrates a cross-sectional view of the stent 820 shown in FIG. 12. FIG. 14 illustrates the membrane 850 of the knitted stent 820 as it moves between a relaxed, longitudinally contracted state (shown in FIG. 11) to an elongated state (shown in FIG. 12). FIG. 14 illustrates that the membrane 850 may move between a relaxed state (FIG. 11) to a taut state when in the elongated state (FIG. 12). It can be appreciated that the membrane 850 may define a leak-free, passageway (e.g., channel, lumen, tunnel, etc.) which may permit drainage of bodily substances when in the relaxed state (FIG. 13) and while in the elongated state (FIG. 14).

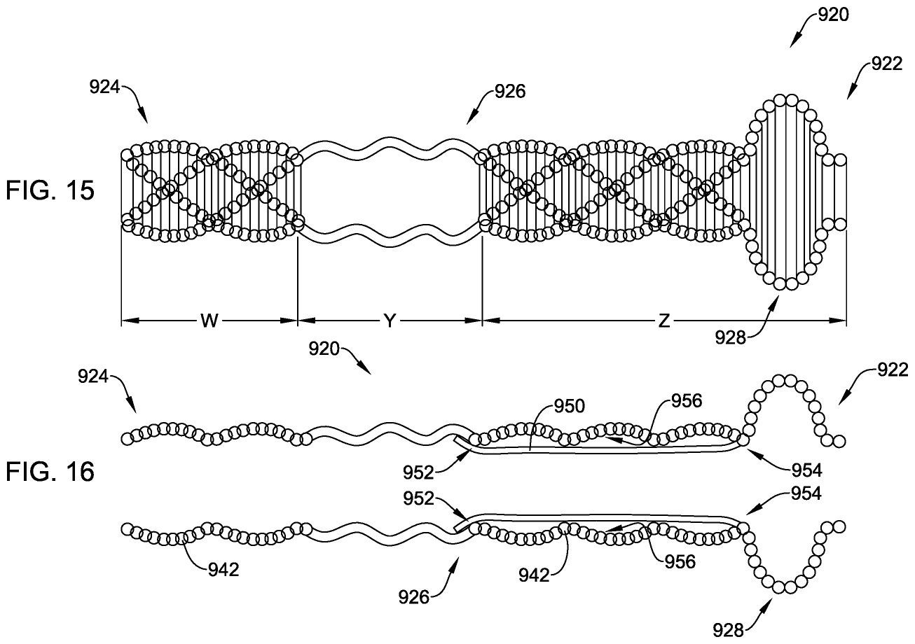

FIG. 15 illustrates another example expandable medical device, namely a stent 920. The stent 920 may be similar in form and function to the stent 820 described herein. The stent 920 may include a first end 922 and a second end 924. The stent 920 may include a lumen extending between the first end 922 and the second end 924. Additionally, FIG. 15 illustrates the stent 920 may include a first knitted region W and a second kitted region Z. FIG. 15 further illustrates that the stent 920 may include a coated region Y positioned between the first knitted region W and the second knitted region Z. The coated region Y may be defined as a region of the stent 920 include knitted stent filaments 942 which are encapsulated in a coating. The coating of the stent filaments 942 along the encapsulated coated region Y may be prevent the region Y of the stent 920 from elongating. It can be appreciated that the first knitted region Y and the second knitted region Z may be permitted to elongate similar to the stent 820 described herein.

FIG. 16 illustrates a cross-sectional view of the stent 820 shown in FIG. 15. FIG. 16 further illustrates that the stent 920 may include a membrane 950 extending along a portion of the inner surface of the knitted stent filaments 942 defining the knitted scaffold of the stent 920. In other words, FIG. 16 illustrates that the stent 920 may include a membrane 950 extending within the inner lumen of the knitted scaffold of the stent 920, whereby the membrane 950 may be attached at various positions along the inner surface of the knitted scaffold of the stent 920.

For example, FIG. 16 illustrates that membrane 950 may be attached to the knitted scaffold of the stent 920 at a first discrete circumferential attachment position 952 and a second discrete circumferential attachment position 954. It can be appreciated that the membrane 950 may be attached around the entire circumference along the inner surface of the knitted scaffold of the stent 920 at both the first discrete circumferential attachment position 952 and the second discrete circumferential attachment position 954. It can be further appreciated from FIG. 16 that the membrane 950 may extend away from the inner surface 956 of the knitted filament members 942 (which form the knitted scaffold of the stent 920) along the portion of the stent structure which extends between the first discrete circumferential attachment position 952 and the second discrete circumferential attachment position 954.

Additionally, the knitted stent filaments 942 along the retention member 928 of the knitted scaffold of the stent 920 may include a coating which spans across interstices of the knitted scaffold of the stent 920 along the retention member 928. The coating may prevent tissue ingrowth along their respective surfaces and interstices therebetween. Additionally, the coating which spans the knitted filaments 942 defining the retention member 928 may provide a leak-free surface. Accordingly, the coated knitted filaments 942 defining the retention member 928 together with the membrane 950 may define a leak-free, passageway (e.g., channel, lumen, tunnel, etc.) which may permit drainage of bodily substances (e.g., bile) from one anatomical organ (e.g., liver) to another anatomical organ (e.g., stomach).

Further, it can be appreciated that tissue may be permitted to grow around, between, through, within, etc. those knitted filament members 942 of the knitted scaffold of the stent 920 in which the membrane 950 is not attached. In other words, FIG. 16 illustrates multiple “tissue ingrowth regions” defined along the medial region 926 of the knitted scaffold of the stent 920. Some tissue ingrowth regions may be defined as the space between the inner surface 956 of the tubular scaffold of the stent 920 and the outer surface of the membrane 950 extending between discrete attachment points 952/954. As discussed herein, uncovered or bare portions of the knitted scaffold of the stent 920 may provide structures that promote tissue ingrowth to anchor the stent 920 in place and reduce the risk of stent migration.

It can be appreciated that for any of the example stent configurations described herein, coating and/or encapsulating a greater proportion of the stent strut members may result in a less flexibility stent design. In other words, the example stents disclosed herein illustrate stent designs having different proportions (e.g., ratios) of uncovered (e.g., bare) verses covered (e.g., with a membrane, coating, etc.) stent strut members. It can be appreciated that these designs may vary in flexibility, with the stents having a lower percentage of covered stent strut members being more flexible.

It can be appreciated that, in addition to the HGS procedure described herein, the example stents described herein may be used in other medical procedures. For example, the stent designs described herein may be utilized in a gastrojejunal (GJ) bypass procedure. One GJ procedure involves inserting one end of a stent into the stomach wall and the other end of the stent into a distal portion of the small intestine. The stent creates an anastomosis between the small intestine and the stomach, effectively rerouting the stomach contents directly into the small intestine. As both the stomach wall and intestine experience peristaltic motion, this would be considered a highly motile application, which may benefit from the stent, where the bypass can be achieved while allowing an adaptable channel which retains its diameter throughout peristalsis.

U.S. Provisional Patent Application No. 63/246,376, filed Sep. 21, 2021, and U.S. Patent Application No. 17,941.867, filed Sep. 9, 2022, are herein incorporated by reference in their entirety for any and all purposes. These applications describe stents for implantation in body lumens and associated methods.

The stents, delivery systems, and the various components thereof, may be made from a metal, metal alloy, polymer (some examples of which are disclosed below), a metal-polymer composite, ceramics, combinations thereof, and the like, or other suitable material. Some examples of suitable metals and metal alloys include stainless steel, such as 304V, 304L, and 316LV stainless steel; mild steel; nickel-titanium alloy such as linear-elastic and/or super-clastic Nitinol; other nickel alloys such as nickel-chromium-molybdenum alloys, nickel-copper alloys, nickel-cobalt-chromium-molybdenum alloys, nickel-molybdenum alloys, other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys; platinum enriched stainless steel; titanium; combinations thereof; and the like; or any other suitable material.

Some examples of suitable polymers for the stents or delivery systems may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex® high-density polyethylene, Marlex® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like.

In at least some embodiments, portions or all of the stents or delivery systems may also be doped with, made of, or otherwise include a radiopaque material. Radiopaque materials are generally understood to be materials which are opaque to RF energy in the wavelength range spanning x-ray to gamma-ray (at thicknesses of <0.005″). These materials are capable of producing a relatively dark image on a fluoroscopy screen relative to the light image that non-radiopaque materials such as tissue produce. This relatively bright image aids the user of the stents or delivery systems in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the stents or delivery systems to achieve the same result.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The disclosure's scope is, of course, defined in the language in which the appended claims are expressed.

What is claimed is:

1. An expandable medical device, comprising:
a tubular scaffold, the tubular scaffold including an inner surface, an outer surface, a proximal end region, a distal end region, a lumen extending from the proximal end region to the distal end region, and a portion of the tubular scaffold defining a retention member extending radially away from the outer surface, wherein the retention member has a distally facing surface and a proximally facing surface; and
a membrane disposed within the lumen of the tubular scaffold;
wherein the membrane is secured to the inner surface of the tubular scaffold at a first circumferential attachment region, and wherein the membrane is secured to the inner surface of the tubular scaffold at a second circumferential attachment region;
wherein the membrane is unattached to the inner surface of the tubular scaffold between the first circumferential attachment region and the second circumferential attachment region to define a tissue ingrowth region between the inner surface of the tubular scaffold and an outwardly-facing surface of the membrane.

2. The medical device of claim 1, wherein the membrane is configured to maintain a passageway therethrough.

3. The medical device of claim 2, wherein the tissue ingrowth region extends circumferentially around the inner surface of the tubular scaffold.

4. The medical device of claim 3, wherein the membrane is formed from an elastic material.

5. The medical device of claim 1, wherein the membrane is designed to permit tissue ingrowth between the inner surface of the tubular scaffold and an outwardly-facing surface of the membrane.

6. The medical device of claim 5, wherein the first circumferential attachment region is secured to the inner surface of the tubular scaffold along the distal end region and wherein the second circumferential attachment region is secured to the inner surface of the tubular scaffold at a position distal to the retention member.

7. The medical device of claim 6, wherein the tubular scaffold includes interstices extending from the outer surface of the tubular scaffold to the inner surface of the tubular scaffold, and wherein the membrane spans the interstices of the portion of the tubular scaffold which defines the retention member.

8. The medical device of claim 7, wherein the tubular scaffold includes interstices extending from the outer surface of the tubular scaffold to the inner surface of the tubular scaffold, and wherein the membrane encapsulates the interstices of the portion of the tubular scaffold which defines the retention member.

9. The medical device of claim 5, wherein the first circumferential attachment region is secured to the inner surface of the tubular scaffold along the distal end region and wherein the second circumferential attachment region is secured to the inner surface of the tubular scaffold at a position proximal to the retention member.

10. The medical device of claim 9, wherein the tubular scaffold includes interstices extending from the outer surface of the tubular scaffold to the inner surface of the tubular scaffold, wherein tissue is permitted to grow through the interstices of the portion of the tubular scaffold between the first circumferential attachment region and the second circumferential attachment region.

11. The medical device of claim 5, wherein the distal end region of the tubular scaffold further includes a flared portion.

12. The medical device of claim 11, wherein the first circumferential attachment region is secured to the inner surface of the tubular scaffold at a position proximal to the flared portion and wherein the second circumferential attachment region is secured to the inner surface of the tubular scaffold at a position distal to the retention member.

13. The medical device of claim 12, wherein the membrane is in direct contact with the inner surface of the portion of the tubular scaffold defining the retention member.

14. The medical device of claim 13, wherein the flared portion includes interstices extending from the outer surface of the tubular scaffold to the inner surface of the tubular scaffold, wherein the flared portion is devoid of the membrane such that tissue is permitted to grow through the interstices of the tubular scaffold along the flared portion.

15. The medical device of claim 14, wherein the retention member has a diameter, wherein the flared portion has a diameter, and wherein the diameter of the retention member is greater than the diameter of the flared portion.

16. The medical device of claim 1, wherein the distally facing surface of the retention member is substantially parallel to the proximally facing surface of the retention member.

17. The medical device of claim 1, wherein the membrane is further secured to the inner surface of the tubular scaffold at a plurality of spaced-apart discrete attachment points positioned between the first circumferential attachment region and the second circumferential attachment region.

18. The medical device of claim 17, wherein regions of the membrane between the plurality of spaced-apart discrete attachment points are radially spaced from the inner surface of the tubular scaffold, creating a plurality of tissue ingrowth regions positioned between the first circumferential attachment region and the second circumferential attachment region.

19. An expandable medical device, comprising:
a tubular scaffold, the tubular scaffold including an inner surface, an outer surface, a proximal end region, a distal end region including a flared portion, a lumen extending from the proximal end region to the distal end region, and a retention member extending radially away from the outer surface, wherein the retention member has a distally facing surface positioned substantially parallel to a proximally facing surface; and
a membrane disposed within the lumen of the tubular scaffold, and wherein the membrane is secured to the inner surface of the tubular scaffold at a first circumferential attachment region, and wherein the membrane is secured to the inner surface of the tubular scaffold at a second circumferential attachment region;
wherein the membrane is configured to maintain a passageway therethrough.

20. An expandable medical device, comprising:
a tubular scaffold, the tubular scaffold including an inner surface, an outer surface, a proximal end region, a distal end region including a flared portion, a lumen extending from the proximal end region to the distal end region, and a retention member extending radially away from the outer surface, wherein the retention member has a distally facing surface positioned substantially parallel to a proximally facing surface; and
a membrane disposed within the lumen of the tubular scaffold, and wherein the membrane is secured to the inner surface of the tubular scaffold at a first circumferential attachment region, and wherein the membrane is secured to the inner surface of the tubular scaffold at a second circumferential attachment region;
wherein the flared portion includes interstices extending from the outer surface of the tubular scaffold to the inner surface of the tubular scaffold, wherein the flared portion is devoid of the membrane such that tissue is permitted to grow through the interstices of the tubular scaffold along the flared portion.
